(19) United States
(12) Patent Application Publication
(10) Pub. No.: US 2017/0326066 A1
(43) Pub. Date: Nov. 16, 2017

(54) METHOD OF TREATING HYPERTENSION

(71) Applicants: Ezra Pharma LLC, Little Rock, AR (US); Rubicon Research Private Limited, Mumbai (IN)

(72) Inventors: Joseph A. Fix, Lawrence, KS (US); Shirish A. Shah, Phoenix, AZ (US); Pratibha S. Pilgaonkar, Mumbai (IN); Anilkumar S. Gandhi, Mumbai (IN)

(73) Assignees: Ezra Pharma LLC, Little Rock, AR (US); Rubicon Research Private Limited, Mumbai (IN)

(21) Appl. No.: 15/154,850

(22) Filed: May 13, 2016

(51) Int. Cl. A61K 9/00; A61K 9/28; A61K 9/20; A61K 31/41

(52) U.S. Cl. CPC A61K 9/0065; A61K 31/41; A61K 9/2866; A61K 9/284; A61K 9/282; A61K 9/2009; A61K 9/2054; A61K 9/2027; A61K 9/2013; A61K 9/2813

(57) ABSTRACT

The present invention relates to methods and composition for treatment of hypertension. Particularly the present invention provides a method of treating hypertension comprising orally administering once a day to a human patient in need thereof the compound valsartan in an extended release dosage form, wherein said extended release valsartan dosage form reduces mean systolic and diastolic blood pressure in a patient during a time period of about 20 hours to about 24 hours after administration to a greater extent than an immediate release formulation of valsartan.

### FIELD OF THE INVENTION

The present invention relates to methods and composition for treatment of hypertension. Particularly the present invention relates to methods and extended release pharmaceutical compositions of valsartan for treatment of hypertension. Further the present invention provides a method of treating hypertension comprising orally administering once a day to a human patient in need thereof the compound valsartan in an extended release dosage form, wherein the extended release valsartan dosage form reduces mean systolic and diastolic blood pressure in a patient during a time period of about 20 hours to about 24 hours after administration to a greater extent than an immediate release formulation of valsartan. Furthermore, the present invention provides a method of treating hypertension comprising administering once a day to a patient in need thereof the compound valsartan in an extended release dosage form, wherein the extended release valsartan dosage form exhibits more than 25% reduction in the mean diastolic blood pressure when compared to immediate release formulation of valsartan during a time period of about 20 hours to about 24 hours after administration.

### BACKGROUND OF THE INVENTION

Cardiovascular disease remains the most common cause of death in the United States. Most cardiovascular events are attributed to high blood pressure. Hypertension, the most important cardiovascular risk factor, currently affects approximately 75 million Americans. This number is expected to increase dramatically over the next 10-15 years as the population ages. Epidemiologic data shows that only about 50% of hypertensive subjects are managing the disease to a blood pressure (BP) level of <140/90 mmHg indicating the need for more effective therapies.

The influence of circadian rhythms in cardiovascular disease is well established. It is also known that heart rate and blood pressure normally peak during the morning hours and reach a nadir in the late evening, around bedtime. The incidence of myocardial infarction, stroke, sudden cardiac death, and myocardial ischemia increases during the early morning hours. Angina attacks occur in a diurnal cycle; their occurrence is common in the hours shortly after an individual begins activity or after waking. In case of cardiovascular agents such as antihypertensive agents, the optimal levels of active have to be maintained throughout the circadian rhythms so that the heart rate, heart rhythm, and blood pressure is maintained reducing the chances of stroke, sudden cardiac death, and other myocardial ischemia diseases.

Angiotensin receptor blockers (ARBs) are well known antihypertensives that work by blocking angiotensin II thereby dilating the arterial vessels and reducing the blood pressure, and making it easier for the heart to pump. Valsartan, a selective ARB, is a well-known antihypertensive agent. Valsartan is rapidly absorbed from the gastrointestinal tract after oral administration. The absolute bioavailability of valsartan is about 25% (10-35%). This relatively low bioavailability of valsartan is primarily due to its poor solubility in the acid milieu of the gastrointestinal tract. Valsartan is an acid, and therefore, has good solubility at pH >5 and low solubility in acidic conditions of the gastrointestinal (GI) milieu. Valsartan becomes ionized in small intestine and hence cannot get absorbed in ionized form. Valsartan typically gets absorbed relatively slowly with Tₘₐₓ in the range of 2-4 hours, however following Cₘₐₓ the plasma levels starts reducing reaching to very low level by about 10-12 hours considering that the half-life of valsartan is about 6 hours. The desired antihypertensive effect therefore may not be observed when it is much needed in accordance with circardian rhythm. With the marketed immediate release formulation Diovan® there is an 80% inhibition of the pressor effect of angiotensin II infusions at valsartan peak plasma concentration (Tₘₐₓ˜3 hour) and at 24 hour there is 30% reduction in the pressor effect. Also the peak plasma concentration of valsartan is reduced to approximately 65% by 12 hours. Due to such fluctuations in the plasma levels of the active, Diovan®, normally prescribed in once-a-day regimen for the treatment of hypertension, may therefore be less optimal. Rapid decline in plasma levels of the marketed immediate release formulation Diovan® results in lack of effective blood pressure control over the entire 24 hour time period and hence makes the product short acting. This creates treatment gaps and such gaps (especially in early morning hours which are more vulnerable) increase potential of precipitating adverse cardiovascular events.

In practice, management of hypertension with Diovan® monotherapy often proceeds to twice a day dosing and/or combination therapy with a second pharmacologic agent such as hydrochlorothiazide due to inadequate blood pressure control over a 24 hour time period. Hypertension monotherapy with immediate release formulations of valsartan therefore lacks effective blood pressure control over a period of 24 hours and may cause increased number of non-responders to the therapy. Treatment with Diovan® does not provide true 24 hour blood pressure control, and tends to lose efficacy towards the end of the dosing interval, which coincides with the time that patients are at their greatest risk of precipitating adverse cardiovascular events. Patient compliance with once a day formulations, efficacy of the formulation and duration of action thereof therefore are some parameters that need to be considered for the treatment of hypertension.

US Patent Publication 20100233253 discloses an extended release gastro-retentive drug delivery system of valsartan containing a release portion containing valsartan, a gastro-retentive portion for retaining the drug delivery system in the stomach and an optional secondary portion for delivering a secondary pulse of Valsartan. WO2009084040 relates to controlled release formulation of angiotensin receptor blockers (ARB) prepared by incorporating pharmaceutically effective amounts of solubilized ARB into a gastroretentive dosage form for once a day administration. These publications, however, do not disclose providing an effective 24 hour blood pressure control.

A need thus exists for a method of treating hypertension that provides 24 hour blood pressure control.

The present inventors have surprisingly found that administration of extended release formulation of valsartan that provides greater extent of reduction in mean systolic and diastolic blood pressure in a patient during a time period of about 20 hours to about 24 hours after administration than an immediate release formulation of valsartan is able to provide an improved 24 hour blood pressure control, thereby reducing the risks of adverse cardiovascular events. The present invention further provides method of treating hypertension comprising administering once a day to a patient in need thereof the compound valsartan in an extended release dosage form wherein the extended release dosage form exhibits more than 25% reduction in mean diastolic blood pressure when compared to an immediate release formulation of valsartan during the 20 hour to 24 hour time period after administration.

### SUMMARY OF THE INVENTION

The present invention relates to methods of treatment of hypertension using valsartan formulations. Further the present invention provides a method of treating hypertension comprising orally administering once a day to a human patient in need thereof the compound valsartan in an extended release dosage form, wherein the extended release valsartan dosage form reduces mean systolic and diastolic blood pressure in a patient during a time period of about 20 hours to about 24 hours after administration to a greater extent than an immediate release formulation of valsartan. Furthermore, the present invention provides a method of treating hypertension comprising administering once a day to a patient in need thereof the compound valsartan in an extended release dosage form, wherein the extended release valsartan dosage form exhibits more than 25% reduction in the mean diastolic blood pressure when compared to immediate release formulation of valsartan during a time period of about 20 hours to about 24 hours after administration.

### BRIEF DESCRIPTION OF THE DRAWINGS

FIG. 1 is a graph showing the change in mean diastolic blood pressure (DBP) observed with valsartan extended release tablet formulation of Example 1 as compared to marketed valsartan immediate release formulation (Diovan®) during 24 hour dosing interval and during daytime.

FIG. 2 is a graph showing the change in mean systolic blood pressure (SBP) observed with valsartan extended release tablet formulation of Example 1 as compared to marketed valsartan immediate release formulation (Diovan®) during 24hour dosing interval and during daytime.

FIG. 3 is a graph showing the change in mean SBP and DBP observed with valsartan extended release tablet formulation of Example 1 as compared to marketed valsartan immediate release formulation (Diovan®) during 20 to 24 hours period after administration.

### DETAILED DESCRIPTION OF THE INVENTION

The present invention provides methods of treating hypertension by administering extended release formulation of valsartan exhibiting an extended vasodilation effect over a time period of 24 hours. In one embodiment, the present invention provides methods for obtaining an improved control over blood pressure over the dosing interval of 24 hours when compared to treatment of hypertension with immediate release formulations of valsartan.

In a further embodiment, the present invention provides a method of treating hypertension comprising orally administering once a day to a human patient in need thereof the compound valsartan in an extended release dosage form, wherein the extended release valsartan dosage form reduces mean systolic and diastolic blood pressure in a patient during a time period of about 20 hours to about 24 hours after administration to a greater extent than an immediate release formulation of valsartan. In another embodiment, the present invention provides a method of treating hypertension comprising administering once a day to a patient in need thereof the compound valsartan in an extended release dosage form, wherein the extended release valsartan dosage form exhibits more than 25% reduction in the mean diastolic blood pressure when compared to immediate release formulation of valsartan during a time period of about 20 hours to about 24 hours after administration.

In one embodiment, the present invention provides a method of treating hypertension comprising orally administering once a day to a human patient in need thereof the compound valsartan in an extended release dosage form, wherein the extended release valsartan dosage form reduces mean systolic and diastolic blood pressure in a patient over 24 hour dosing interval to a greater extent than a placebo formulation. In another embodiment, the present invention provides a composition that exhibits more than 2 mmHg reduction in blood pressure when compared to placebo formulation during the 24 hour dosing interval and methods of treating hypertension using the same.

In one embodiment, method of treating hypertension comprises administration of extended release formulation of valsartan that provides greater absorption of valsartan and more effective and even plasma levels over 24 hour dosing interval not achievable by the currently marketed immediate release formulation. In a further embodiment, the method of treatment of hypertension of the present invention prevents treatment gaps and the chances of precipitating adverse events otherwise observed with immediate release formulations of valsartan.

The term “composition” or “formulation” or “dosage form” has been employed interchangeably for the purpose of the present invention and means that it is a pharmaceutical formulation which is suitable for administration to a patient. For the purpose of the present invention, the terms “controlled release” or “sustained release” or “extended release” or “modified release” or “prolonged release” have been used interchangeably and mean broadly that the active agent is released at a predetermined rate that is different or slower than immediate release of the active agent. In one embodiment, extended release means release of valsartan from the dosage form at such a rate that when a once-a-day dose of the active agent is administered plasma concentrations (levels) of the drug are maintained within the therapeutic range but below toxic levels over a period of time of about 24 hours.

The term “blood pressure” as employed herein means the pressure exerted by circulating blood upon the walls of blood vessels. It is generally referred to as arterial pressure of the systemic circulation. It is measured in millimeters of mercury (mmHg).

The term “systolic blood pressure (SBP)” as used herein means the maximum arterial pressure of the left ventricle of the heart when the heart is contracting while pumping blood. A normal systolic blood pressure in humans is generally between 90-119 mmHg. A systolic blood pressure of 120 mmHg to 139 mmHg is referred as prehypertension or borderline high blood pressure. Even people with prehypertension are at a higher risk of developing heart disease.

The term “diastolic blood pressure (DBP)” as used herein means the minimum arterial pressure during relaxation and dilatation of the ventricles of the heart when the ventricles fill with blood i.e. when the heart is at rest between beats. A normal diastolic blood pressure in humans is generally between 60-79 mmHg. A diastolic blood pressure between 80 mmHg and 89 mmHg indicates prehypertension.

The term “hypertension” as used herein means a systolic blood pressure number of 140 mmHg or higher and a diastolic blood pressure number of 90 mmHg or higher is considered to be hypertension or high blood pressure.

The term “reduction in blood pressure” as employed herein refers to reduction of blood pressure of at least 0.5 mmHg of either systolic blood pressure or diastolic blood pressure, or both after administration of immediate release or extended release formulation when compared to the baseline value (at time 0) at the start of the treatment. In a further embodiment, reduction of blood pressure is at least 1 mmHg when compared to the baseline value (at time 0) at the start of the treatment.

Reduction in blood pressure is to a greater extent with extended release valsartan formulation than that observed with immediate release valsartan formulation' means the difference in the reduction in blood pressure observed with extended release valsartan formulation is at least 0.05 mmHg more than that observed with immediate release valsartan formulation. In a further embodiment, reduction in blood pressure observed with extended release valsartan formulation is at least 0.1 mmHg more than that observed with immediate release valsartan formulation.

The term “single dose/dosing” means that the patient has received a single dose of the drug formulation and the drug plasma concentration has not achieved steady state. The term “mean”, when preceding a pharmacokinetic value represents the arithmetic mean value of the pharmacokinetic value taken from a population of patients unless otherwise specified (e.g. geometric mean). The term “AUC” as used herein, means area under the plasma concentration time curve, as calculated by the trapezoidal rule over the complete 24-hour interval. AUC may be further defined to refer to specific time periods relative to administration of the drug. For example, AUC₂₀₋₂₄ refers to area under the plasma concentration time curve over the period of 20 hours to 24 hours.

In a further embodiment, the extended release formulation employed in the present invention for the treatment of hypertension comprises valsartan and at least one pharmaceutically acceptable excipient.

In one embodiment, valsartan may be present in the formulation employed in the method of the present invention in crystalline, substantially crystalline, amorphous, substantially amorphous, or dissolved form and the like or any combinations thereof. The crystalline form may have different polymorphs. All different polymorphs, solvates, hydrates, salts are within the purview of this invention. Also included within the scope of the present invention are the salts, esters, amides, prodrugs, active metabolites, analogs, and the like of valsartan. In one embodiment, valsartan in the formulations employed in the method of the present invention in an amount typically ranging from about 40 mg to about 640 mg. In a further embodiment, the amount of valsartan in the formulations employed in the method of the present invention is from about 40 mg to about 320 mg. In another embodiment, the amount of valsartan in the formulations employed in the method of the present invention is from about 80 mg to about 320 mg. In one embodiment, valsartan may be present in an amount from about 1% to about 80% by weight of the composition. In one embodiment, the valsartan is present in an amount from about 2% to about 70% by weight of the composition. In another embodiment, the valsartan is present in an amount from about 5% to about 50% by weight of the composition.

In another embodiment, the extended release formulation employed in the method of treatment of hypertension of the present invention comprises valsartan, at least one solubilizer, and at least one pharmaceutically acceptable excipient. In a further embodiment, the extended release formulation employed in the method of the present invention comprises valsartan, at least one solubilizer, at least one swelling polymer and at least one pharmaceutically acceptable excipient. In another embodiment, extended release formulation employed in the methods of the present invention may comprise substantially solubilized valsartan, and at least one pharmaceutically acceptable excipient. In a further embodiment, extended release formulation employed in the methods of the present invention may comprise substantially solubilized valsartan, at least one swelling polymer and at least one pharmaceutically acceptable excipient. In a further embodiment, the formulation employed in the methods of the present invention may comprise at least a portion of dissolved valsartan. In another embodiment, the formulation employed in the methods of the present invention may comprise substantially amorphous valsartan.

In one embodiment, one or more solubilizers employed in the compositions used in the methods of the present invention may be polymeric or non-polymeric in nature. In a further embodiment, one or more solubilizers include, but are not limited to, cationic, anionic, zwitterionic or amphiphilic, or nonionic surfactants and the like or any combinations thereof. The ionic surfactants include, but are not limited to, alkylammonium salts; fusidic acid salts; fatty acid derivatives of amino acids, oligopeptides, or polypeptides; glyceride derivatives of amino acids; lecithins or hydrogenated lecithins; lysolecithins or hydrogenated lysolecithins; phospholipids or derivatives thereof; lysophospholipids or derivatives thereof; carnitine fatty acid ester salts; salts of alkylsulfates; fatty acid salts; sodium docusate; acyl lactylates; mono- or di-acetylated tartaric acid esters of mono- or di-glycerides; succinylated mono- or di-glycerides; citric acid esters of mono- or diglycerides; and the like or mixtures thereof. The amphiphilic surfactants include, but are not limited to, d-atocopherylpolyethylene glycol 1000 succinate and d-a-tocopherol acid salts such as succinate, acetate, and the like or mixtures thereof. The non-ionic surfactants include, but are not limited to, fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols or sterol derivatives; polyethylene glycol alkyl ethers; sugar esters; sugar ethers; lactic acid derivatives of mono- or diglycerides; oil soluble vitamins/vitamin derivatives; PEG fatty acid esters; polyglycerized fatty acid; polyoxyethylene-polyoxypropylene block copolymers; transesterification products of a polyol with at least one member of the group consisting of glycerides, vegetable oils, hydrogenated vegetable oils, fatty acids and sterols wherein the commonly used oils are castor oil or hydrogenated castor oil, or an edible vegetable oil such as corn oil, olive oil, peanut oil, palm kernel oil, almond oil and the commonly used polyols include glycerol, propylene glycol, ethylene glycol, polyethylene glycol, sorbitol and pentaerythritol; or mixtures thereof. In another embodiment the one or more solubilizers that may be employed in the compositions used in the methods of the present invention include polyethylene-polyoxypropylene block copolymer (Lutrol® series BASF) and d-alpha-tocopherylpolyethylene glycol 1000 succinate (Vitamin E 25 TPGS® by Eastman) or combinations thereof.

In one embodiment, in the compositions employed in the methods of the present invention valsartan and one or more solubilizers may be employed in different ratios. In one embodiment, the ratio of valsartan to solubilizers may range from about 50:1 to about 1:50. In a further embodiment, the ratio of valsartan to solubilizers is from about 20:1 to about 1:20. In another embodiment, the ratio of valsartan to solubilizer is from about 10:1 to about 1:10. In one embodiment, valsartan is substantially solubilized using one or more solubilizers. In a further embodiment, the term “solubilized” as used herein refers to improved or increased solubility form of valsartan. In another embodiment, the term “substantially solubilized” means valsartan is partially or completely solubilized. The term “completely or partially” as used herein refers to whether the entire or part of the valsartan dose is solubilized. In a further embodiment, the term “substantially solubilized” means more than 50% of valsartan incorporated in the extended release formulation employed in the methods of the present invention is intimately dispersed in a matrix comprising at least one solubilizer. In another embodiment, at least 75% of valsartan incorporated in the extended release formulation employed in the methods of the present invention is intimately dispersed in a matrix comprising at least one solubilizer. In one embodiment, at least 85% of valsartan incorporated in the extended release formulation employed in the methods of the present invention is intimately dispersed in a matrix comprising at least one solubilizer. In another embodiment, at least 95% of valsartan incorporated in the extended release formulation employed in the methods of the present invention is intimately dispersed in a matrix comprising at least one solubilizer. In a further embodiment, the entire amount of valsartan incorporated in the extended release formulation employed in the methods of the present invention is intimately dispersed in a matrix comprising at least one solubilizer.

In a further embodiment, the processes employed for solubilization of valsartan may include but are not limited to melt granulation, solvent treatment, wet granulation, physical mixing or spray drying and the like or combinations thereof. In a further embodiment, the process employed for the solubilization of valsartan in the composition used for treatment of hypertension is melt granulation.

In one embodiment, the swelling polymers in the dry state or in a form that has substantial capacity for water uptake may be employed in the compositions used in the methods of the present invention. Non-limiting examples of such swelling polymers include, but are not limited to, polyalkylene oxides; cellulosic polymers; acrylic acid and methacrylic acid polymers, and esters thereof, maleic anhydride polymers; polymaleic acid; poly(acrylamides); poly(olefinic alcohol)s; poly(N-vinyl lactams); polyols; polyoxyethylated saccharides; polyoxazolines; polyvinylamines; polyvinylacetates; polyimines; starch and starch-based polymers; polyurethane hydrogels;chitosan; polysaccharide gums; alginates; zein; shellac-based polymers; polyethylene oxide, hydroxypropyl cellulose, hydroxypropyl methyl cellulose, hydroxyethyl cellulose, sodium carboxymethylcellulose, calcium carboxymethyl cellulose, methyl cellulose, polyacrylic acid, maltodextrin, pre-gelatinized starch and polyvinyl alcohol, and the like or mixtures thereof. In a further embodiment, one or more swelling polymers employed in the extended release formulations used in the method of the present invention include, but are not limited to, polyethylene oxide, hydroxypropyl cellulose, hydroxypropyl methyl cellulose, hydroxyethyl cellulose, sodium carboxymethyl cellulose, calcium carboxymethyl cellulose, methyl cellulose, polyacrylic acid, maltodextrin, pre-gelatinized starch, polyvinyl alcohol and the like or mixtures thereof. In another embodiment, the weight percent of the swelling polymer in the final compressed dosage form is about 5 to about 95 weight percent.

In another embodiment, the pharmaceutically acceptable excipients employed in the compositions employed in the methods of the present invention include but are not limited to release modifiers, swelling enhancers, acid source, gas generating agents, binders, lubricants, diluents, disintegrants, glidants, colorants, pH modifiers, pore-formers, and the like or mixtures thereof. In a further embodiment, release modifiers employed in the compositions used in the method of the present invention include, but are not limited to, polymeric release retardants, non-polymeric release retardants or any combinations thereof. In one embodiment, release modifiers are employed in the compositions used in the method of the present invention to control the release of valsartan and/or solubilized valsartan. In another embodiment, swelling or non-swelling polymeric release modifiers may be employed in the compositions used in the methods of the present invention. Polymeric release modifiers employed in the compositions used for the methods of the present invention include, but are not limited to, cellulose derivatives; cross-linked polyvinyl pyrrolidone, polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; or any combinations thereof. Cellulose derivatives include, but are not limited to, ethyl cellulose, methylcellulose, hydroxypropylmethylcellulose (HPMC),hydroxypropyl cellulose (HPC), hydroxyethyl cellulose, hydroxymethyl cellulose, hydroxypropylethylcellulose, carboxymethylethyl cellulose, carboxy-ethylcellulose, carboxymethylhydroxyethylcellulose, hydroxyethylmethylcarboxymethyl cellulose, hydroxyethyl methyl cellulose, carboxymethylcellulose (CMC), methylhydroxyethyl cellulose, methylhydroxypropyl cellulose, carboxymethylsulfoethyl cellulose, sodium carboxymethyl cellulose, or combinations thereof. In one embodiment release modifiers of one or more viscosity values may be employed. In another embodiment, the release modifier employed is hydroxypropylmethylcellulose. Non-polymeric release modifiers employed in the compositions used in the method of the present invention include, but are not limited to, fats, oils, waxes, fatty acids, fatty acid esters, long chain monohydric alcohols and their esters or combinations thereof. The amount of release modifier in the dosage form generally varies from about 5% to about 90% by weight of the dosage form.

In one embodiment, the swelling enhancers that may be employed in valsartan extended release composition that are used for method of treatment of hypertension include, but are not limited to, low-substituted hydroxypropyl cellulose, microcrystalline cellulose, cross-linked sodium or calcium carboxymethyl cellulose, cellulose fiber, cross-linked polyvinyl pyrrolidone, cross-linked polyacrylic acid, cross-linked Amberlite resin, alginates, colloidal magnesium-aluminum silicate, corn starch granules, rice starch granules, potato starch granules, pregelatinised starch, sodium carboxymethyl starch and the like or combinations thereof. In another embodiment, the content of the swelling enhancer that may be employed is about 5 to about 90 weight percent of the formulation. At concentration above 5% w/w the non-limiting list of agents listed above function as swelling enhancers and help swelling polymers to swell rapidly. In a further embodiment, acidulants may be employed in the compositions. Non-limiting examples of acidulants that may be employed in the compositions include aliphatic or aromatic, saturated or unsaturated, monobasic acid (monocarboxylic acid), dibasic acid (dicarboxylic acid) or tribasic acid (tricarboxylicacid). In one embodiment of the present invention, the acidulant used in the extended release compositions employed for the method of treatment of the present invention is malic acid, tartaric acid, fumaric acid, maleic acid, aspartic acid or citric acid and the like or any combinations thereof. The acidulants also function as acid source when with the gas generating agent as an effervescent couple. In a further embodiment, the extended release pharmaceutical composition may comprise at least one gas generating agent. The gas generating agents also referred to as effervescent agent aid in the formation of highly porous, preferably honeycombed structure and enhances the buoyancy of the formulation. The gas generating agent employed in the composition is selected from, but not limited to, alkali and alkaline-earth metal carbonates and bicarbonates such as sodium bicarbonate, sodium glycine carbonate, potassium bicarbonate, ammonium bicarbonate, sodium bisulfite, sodium metabisulfite, sodium carbonate, potassium carbonate and the like or combinations thereof. In one embodiment, the gas generating agent is used at concentration from about 0.5 weight % to about 25 weight % of the dosage form. Non-limiting examples of suitable binders that may be employed in the compositions include, but are not limited to, starch, pregelatinized starch, polyvinyl pyrrolidone (PVP), copovidone, cellulose derivatives, such as hydroxypropylmethyl cellulose (HPMC), hydroxypropyl cellulose (HPC), carboxymethyl cellulose (CMC) and their salts and the like or combinations thereof. Non-limiting examples of diluents that may be employed in the compositions include, but are not limited to, starch, dicalcium phosphate, microcrystalline cellulose, lactose monohydrate, dextrate hydrated and the like or combinations thereof. Suitable lubricants that may be employed include, but are not limited to,magnesium stearate, calcium stearate, stearic acid, talc, sodium stearylfumarate and the like or combinations thereof. Non-limiting examples of glidants that may be employed include, but are not limited to, colloidal silica, colloidal silicon dioxide, silica gel, precipitated silica, and the like or combinations thereof. Suitable pore forming agents such as, but not limited to, dextrates, non-GMO dextrates, lactose, sodium chloride, and the like or combinations may be employed in the formulations. Suitable adsorbents that may be used in the formulations, include but are not limited to, silicates such as aluminum magnesium metasilicate, calcium silicate, and the like; microcrystalline celluloses and the like or combinations thereof. Suitable colorants such as, but not limited to, ferric oxide (Sicovit Red 30 E172) may be employed in the compositions used in the method of treatment of the present invention. Disintegrating agents such as, but not limited to, starch, sodium starch glycolate, pregelatinised starch, crosslinked polyvinyl pyrrolidone, cross linked carboxymethylcellulose, or ion exchange resin, may be employed in the composition if required. In one embodiment, disintegrating agents as listed herein above may be employed in the compositions to improve the dissolution.

In a further embodiment, the formulation used in the method of the present invention is in the form of an extended release formulation. In another embodiment the extended release formulation employed in the method of the present invention is in the form of a gastroretentive dosage form. For the purpose of the present invention the term “gastroretentive” or “gastric retention” or “gastroretention” or “retained in upper gastrointestinal tract” when used with respect to the dosage form of the present invention, means that the dosage form or at least a portion thereof remains in the upper gastrointestinal tract including stomach, for about 30 minutes or more. In another embodiment, the gastroretentive dosage form employed in the methods of the present invention of the present invention remains in the upper gastrointestinal tract including stomach, for about 30 minutes to about 12 hours. In another embodiment extended release formulation is in the form of a gastroretentive dosage form. In a further embodiment, gastroretentive dosage forms that are retained in the upper gastrointestinal tract for a prolonged period of time after oral administration and release the active ingredient continuously at a predetermined rate or in a sustained manner are employed for delivering valsartan.

In a further embodiment, the gastroretentive dosage form may be in the form of a monolithic system, an expanding bilayered or multilayered or in-lay system for oral administration which is adapted to deliver the drug at a predetermined rate. In one embodiment, valsartan is incorporated in monolithic matrix type of extended release gastroretentive formulation. In another embodiment, valsartan is incorporated in a bilayered gastroretentive dosage form that consists of a drug layer and a gastroretentive expanding layer wherein the drug is released at a predetermined rate from the drug layer. In a further embodiment pharmaceutical controlled release gastroretentive composition in the form of an expanding bilayered system for oral administration is provided to deliver valsartan from a first layer immediately upon reaching the gastrointestinal tract, and to deliver same or different active, from a second layer, in a sustained manner over a specific time period. The second layer is also adapted to provide expanding nature for the dosage system, thereby making the dosage system have greater retention in the stomach.

In another embodiment, the extended release formulation employed in the methods of treatment of the present invention in the form of a bilayered gastroretentive dosage form exhibits an effective blood pressure control over a period of 24 hours. In another embodiment, the extended release formulation employed in the methods of treatment of the present invention exhibits a better control over systolic and diastolic blood pressure in the high risk 20 to 24 hour period after administration.

In one embodiment, the formulations of the present invention employed for treating hypertension are based on continuous trickling of solubilized drug in the upper gastrointestinal system for longer periods of time thereby extending the absorption phase. In a further embodiment, the sustained release formulations of valsartan are based on controlled release, gastroretention and solubilization concepts surprisingly provide 24 hour blood pressure control.

In yet another illustrative embodiment according to the invention, the extended release formulation employed in the methods of the present invention may be optionally coated. Surface coatings may be employed for aesthetic purposes or for dimensionally stabilizing the dosage form. The coating may be carried out using any conventional technique employing conventional ingredient. A surface coating can, for example, be obtained using a quick-dissolving film using conventional polymers such as, but not limited to, hydroxypropyl methyl cellulose, hydroxypropyl cellulose, carboxymethyl cellulose, ethyl cellulose, polyvinyl alcohol, polymethacrylates or the like or combinations thereof. In one embodiment, preformed coating systems such as, but not limited to, Opadry Clear 03K19229, Opadry 200 Blue 200F105000, Kollicoat® Protect, Kollicoat®Smartseal 30D, Aquarius® MG and the like or combinations thereof may be employed as coatings for the compositions used in the present invention.

Tablets employed in the present invention may vary in shape including, but not limited to, oval, triangle, almond, peanut, parallelogram, pentagonal. It is contemplated within the scope of the invention that the dosage form can be encapsulated. Tablets used in the present invention may be manufactured using conventional techniques of common tableting methods known in the art such as direct compression, dry granulation, wet granulation and extrusion, melt granulation. It is also contemplated within the scope of the invention that any process known in the art suitable for making pharmaceutical compositions in general may be employed for preparation of compositions used in the invention.

In another embodiment, the extended release dosage form employed for the methods of the present invention may be prepared by a process comprising: preparing solubilized valsartan by solubilization using one or more solubilizers; blending said solubilized valsartan with at least one release modifier, at least one swelling polymer and at least one pharmaceutically acceptable excipient; lubricating the blend to form a lubricated blend; compressing the blend to form a monolithic tablet. Furthermore, the extended release dosage form employed for the methods of the present invention may be prepared by a process comprising: preparing solubilized valsartan by solubilization using one or more solubilizers; blending said solubilized valsartan with at least one release modifer and at least one pharmaceutically acceptable excipient, lubricating the blend to form drug layer blend; blending at least one swelling polymer, at least one pharmaceutically acceptable excipient, lubricating the blend to form a gastroretentive layer blend; and compressing the drug layer and the gastroretentive layer to form a bilayered tablet. In one embodiment, the extended compositions employed in the present invention are in the form of bilayered gastroretentive dosage form comprising the active layer and the gastroretentive layer.

The extended release formulations employed for the methods according to the present invention allow for extended release of valsartan. In another embodiment valsartan released over a period of about 24 hours. Further, within the purview of the present invention, are included formulations that comprise a combination of valsartan with other drugs or active agents which may be delivered in an immediate release or modified release manner and the use of such formulations for the treatment of hypertension.

In a further embodiment, the valsartan extended release dosage form used in the methods of the present invention exhibits a ratio of plasma concentrations of the extended release formulation to immediate release formulation of greater than 1 over a time period of 8 to 24 hours after administration in a single dose human pharmacokinetic study.

Without being bound to any theory it is believed that valsartan undergoes irreversible binding with AT1 receptor subtype and drug release from the receptor binding sites is a slow and gradual phenomenon. With the immediate release Diovan formulation this binding phenomenon extends the blood pressure lowering effect for only a very few hours even after plasma concentration has reached towards sub-therapeutic level. It is believed that the method of treatment of hypertension using extended release formulation of valsartan discussed in the present invention provides extended absorption phase that maintains a continuous stream of the active agent valsartan at the AT1 receptor subtype binding sites for prolonged period of time, thereby sustaining blood pressure lowering effect and supporting true once a day administration.

In one embodiment prospective, randomized, open label blinded endpoint (PROBE), crossover study comparing the safety, efficacy, and duration of action of valsartan extended release tablets 160 mg once-daily with Diovan® 160 mg once-daily in patients with stage I and stage II hypertension is conducted to compare the reduction in mean 24-hour systolic blood pressure (SBP) in hypertensive patients treated with valsartan extended release tablets160 mg or with Diovan® 160 mg as measured by Ambulatory Blood Pressure Monitoring (ABPM).

In another embodiment, following 21 days of treatment patients treated in the above mentioned study with both valsartan extended release and Diovan exhibited significant reductions in systolic and diastolic blood (DBP and SBP respectively) compared to baseline levels. In another embodiment, the present invention provides method of treating hypertension by administering to a patient in need thereof extended release valsartan dosage form, wherein the dosage form exhibits numerically greater reduction in mean 24 hour DBP and mean daytime DBP in patients when treated with extended release valsartan formulations compared to when on treatment with Diovan®. In another embodiment, the present invention provides method of treating hypertension by administering to a patient in need thereof extended release valsartan dosage form, wherein the dosage form exhibits numerically greater reduction in patients in mean SBP and mean DBP measured between 20-24 hours compared to when on treatment with Diovan®. In another embodiment, the present invention provides method of treating hypertension comprising administering extended release formulation of valsartan that is well tolerated and provides better blood pressure control than Diovan®, particularly toward the end of treatment (20-24 hours) when patients are most at risk for cardiovascular events.

In another embodiment, the present invention provides a method of treating hypertension comprising orally administering once a day to a human patient in need thereof valsartan in an extended release dosage form, wherein the extended release valsartan dosage form reduces mean systolic and diastolic blood pressure in a patient during the 20-24 hour time period after administration to a greater extent than the immediate release formulation. In one embodiment, the present invention provides a method of treating hypertension comprising orally administering once a dayto a human patient in need thereof valsartan in an extended release dosage form, wherein the extended release valsartan dosage form reduces mean systolic and diastolic blood pressure in a patient during the 24 hour dosing interval to a greater extent than placebo formulation. In a further embodiment the present invention provides a composition that exhibits more than 25%reduction in the mean diastolic blood pressure when compared to immediate release formulation during the 20-24 hour time period after administration. In a further embodiment the present invention provides a method of treating hypertension comprising administering a composition that exhibits more than 2 mmHg reduction in the mean systolic and diastolic blood pressure when compared to placebo formulation during the 24 hour dosing interval. In another embodiment the present invention provides a method of treating hypertension comprising administering a composition that exhibits more than 3 mmHg reduction in the mean systolic and diastolic blood pressure when compared to placebo formulation during the 20-24 hour time period after administration. In a further embodiment the present invention provides a method of treating hypertension comprising administering a the extended release valsartan dosage form that exhibits more than 4 mmHg reduction in mean systolic blood pressure when compared to placebo formulation during 20-24 hour period after administration.

In another embodiment, a method of treating hypertension comprises administering once a day to a patient in need thereof the compound valsartan in an extended release dosage form wherein the extended release valsartan dosage form exhibits more than 3 mmHg reduction in mean systolic and diastolic blood pressure when compared to placebo formulation during the 20-24 hour dosing interval; and wherein the extended release dosage form provides plasma concentration of valsartan greater than that provided by an immediate release formulation of valsartan over a time period of 20 to 24 hours after administration in a single dose human pharmacokinetic study.

In another embodiment, a method of treating hypertension comprises administering once a day to a patient in need thereof the compound valsartan in an extended release dosage form wherein the extended release valsartan dosage form exhibits more than 4 mmHg reduction in mean systolic blood pressure when compared to placebo formulation during the 20-24 hour dosing interval; and wherein the extended release dosage form provides plasma concentration of valsartan greater than that provided by an immediate release formulation of valsartan over a time period of 20 to 24 hours after administration in a single dose human pharmacokinetic study.

In a further embodiment, the method of treating hypertension of the present invention may be employed for treating patients with condition of isolated diastolic hypertension. In another embodiment, the method of treatment of hypertension of the present invention using extended release formulation of valsartan provides higher diurnal to nocturnal blood pressure ratio, thereby normalizing the circardian rhythm providing better end organ protection (for e.g. kidney and cardiovascular system). In a further embodiment, the present method of treatment provides a blood pressure lowering effect that is less dependent on the administration time. By providing consistent drug concentration at the receptor site; the present method of treatment exhibits more consistent therapeutic response by reducing number of non-responders and reverse responders. In another embodiment the formulation of the present invention may be taken with a meal, thereby enabling patients to dose in the morning with breakfast simplifying dosing in that it can be dosed in the morning with a breakfast, which tends to improve patient compliance. In a further embodiment, the method of treating hypertension comprises administering to a patient in need thereof an extended release valsartan dosage form that exhibits more than 3 mmHg reduction in the mean systolic and diastolic blood pressure when compared to placebo formulation during the 20-24 hour time period after administration and may be taken with meal, thereby enabling patients to dose in the morning with breakfast simplifying dosing.

While the present invention has been described in terms of its specific embodiments, certain modifications and equivalents will be apparent to those skilled in the art and are intended to be included within the scope of the present invention. The invention is further illustrated by the following examples, which are for illustrative purposes and should not be construed as limiting the scope of the invention in any way.

### EXAMPLES

### Example 1

This example describes extended release valsartan formulation in Table 1.

TABLE 1

| | |
|---|---|
| Ingredients | mg/unit |

Core Tablets
Active layer

| | |
|---|---|
| Valsartan | 160 |
| Vitamin E polyethylene glycol | 80 |
| succinate | |
| Poloxamer | 80 |
| Microcrystalline Cellulose | 135 |
| Hydroxypropyl methylcellulose | 110 |
| Calcium Silicate | 120 |
| Crospovidone | 35 |
| Fumaric Acid | 78 |
| Dextrates | 60 |
| Colloidal Silicon Dioxide | 10 |
| Magnesium Stearate | 20 |
| Ferric Oxide | 2 |

Gastroretentive layer

| | |
|---|---|
| Polyethylene Oxide | 119 |
| Hydroxypropyl methyl cellulose | 119 |
| Hydroxyethyl Cellulose | 59 |
| Crospovidone | 120 |
| Microcrystalline Cellulose | 29 |
| Polyvinylpyrrolidone | 33 |
| 1-vinyl-2-pyrrolidone and vinyl acetate | 13 |
| copolymer | |
| Sodium Bicarbonate | 33 |
| Anhydrous Citric Acid | 10 |
| Magnesium Stearate | 5 |
| Isopropyl Alcohol | q.s. |
| Purified Water^{#} | q.s. |
| Total | 1430 |

Coating System

| | |
|---|---|
| Polyvinyl alcohol-based Opadry 200 | 45 |
| Blue 200F105000 | |
| Purified water^{#} | q.s. |
| Excipients (imprinting material) | |
| Opacode Black S-1-17823 | 0.104 |
| Isopropyl alcohol^{#} | q.s. |
| Total | 1490 |

^{#}expelled during manufacturing process, not part of the final product

Process of Preparation: Valsartan was added to molten poloxamer and vitamin E polyethylene glycol succinate in a low shear mixer and mixed well. A part of microcrystalline cellulose, calcium silicate, fumaric acid and crospovidone was added to above mass and mixed further to get a homogeneous blend. All other ingredients were added to above mass and granulated to obtain granules of valsartan. These granules were then blended with other excipients except lubricant. The granules were then lubricated using magnesium stearate and compressed to form active layer blend. Povidone was dissolved in IPA: water mixture with overhead stirring. A part of polyethylene oxide, a part of hydroxyl propyl methyl cellulose, hydroxyethyl cellulose, a part of crospovidone, microcrystalline cellulose were passed through the sieve and dry mixed in rapid mixer. The binder solution was added to the dry mix and the mass was granulated and subsequently dried in a fluidized bed dryer to get desired loss on drying. Sized dried granules were blended with all other excipients including lactose, microcrystalline cellulose, sodium bicarbonate and citric acid. The granules were then lubricated using magnesium stearate to form gastroretentive layer blend. A core bilayer gastroretentive tablet of valsartan was prepared by compressing the active layer and the gastroretentive layer.

The core tablets of valsartan were further coated by aqueous coating system comprising polyvinyl alcohol-based Opadry 200 Blue 200F105000 to achieve weight gain level of 4% and then imprinted.

### Example 2

This example summarizes a clinical study comparing effects of valsartan extended release formulation of Example 1 and immediate release formulation Diovan® on hypertension and blood pressure control.

The subjects selected for the study were individuals with clinically established cases of hypertension, male or female, between the age of 56.4±8.4 years, with BMI 31.8±6.0 kg/m², with mean daytime systolic blood pressure (SBP) (08:00-16:00)145±9.2 mmHg with mean daytime diastolic blood pressure (DBP) (08:00-16:00) 90.4±8.7 mmHg and no diagnosed complications. Any previous antihypertensive drugs used by the subjects were discontinued under medical supervision.

The study was a prospective, randomized, open label, blinded endpoint (PROBE) crossover design study. Following initial screening, all the 39 subjects entered a 21 days single blind placebo washout period. Patients, who had a SBP ≧150 and ≦180 mmHg, as measured on an Omron device, were subjected to Ambulatory blood pressure monitoring (ABPM).To obtain baseline blood pressure, 30 hours of ABPM was obtained. Within 48 hours after completing the ABPM, qualifying subjects entered the PK unit where they were randomized to receive either Valsartan extended release (ER) tablets160 mg once-daily or Diovan® 160 mg once-daily in the morning with breakfast for 21 days. A single blind placebo washout period of 21 days to replace treatment was used before switching to the next product as patients were evaluated for both the products. Upon discharge, subjects received 21 days of the assigned investigational product (IP) and then returned for 30 hours of ABPM, followed by a 24-hour PK. Subjects then completed a 21-day washout period with weekly OMRON blood pressure checks.

Subjects commenced Phase B of the study with a 24-hour PK unit visit and were crossed over to receive the alternate IP. Upon discharge from the PK unit the next morning, they received 3 weeks of the assigned, crossed-over IP and returned for 30 hours of ABPM, followed by a concluding 24-hour PK unit visit. Thus, an initial ABPM before each product regimen and a final ABPM at the end of each product regimen was done.

The values of these ABPM were used to calculate the change in mean DBP and mean SBP. Of the 39 patients that were enrolled in this study, 33 completed all study activities. All the ABPM values were statistically analyzed, using changes from baseline values (0-time) to the end of study according to analyses of variance. This data was compared among the test products. During the study, physical parameters such as body weight, body mass index, height, medications, and diet were measured and recorded for each subject. Any significant change, like sudden hypotension episodes were managed by reducing the test products on which the subjects were maintained, as recommended by the study physician. Drugs were well tolerated.

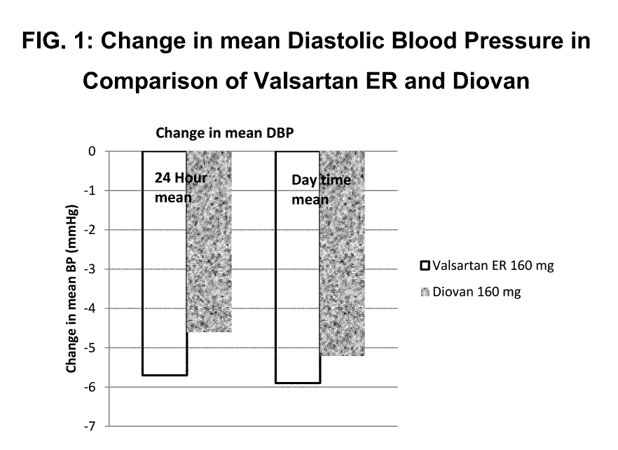

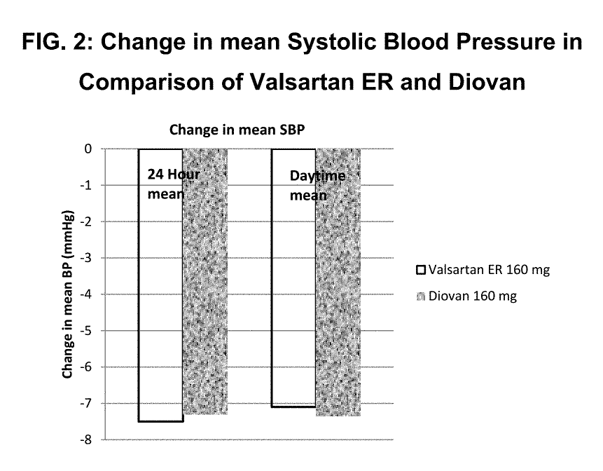

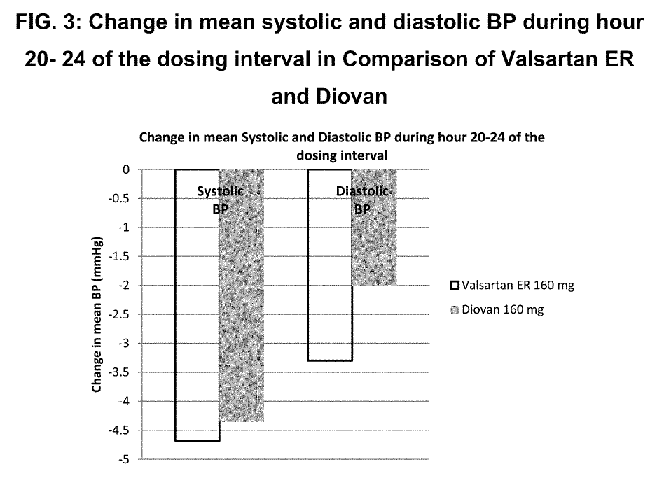

The test results are depicted in FIGS. 1, 2 and 3.

These test results indicate beneficial antihypertensive effects of similar doses of valsartan ER as compared to Diovan® immediate release formulation in patients with hypertension. The patients treated with both valsartan ER and Diovan® had significant reductions in SBP and DBP compared to baseline levels. Patients had numerically greater reduction in mean 24 hour DBP and mean daytime DBP when treated on Valsartan ER compared to when on treatment with Diovan®. Reductions in mean 24 hour SBP were greater with the treatment with valsartan ER and reduction in mean day time SBP were similar when patients treated with each of the drugs. Patients had numerically greater reduction in mean SBP and mean DBP measured between hours 20-24 when treated with Valsartan ER compared to when they were treated with Diovan®.

The above study therefore shows that better blood pressure control was obtained when patients were administered valsartan ER formulation. Valsartan extended release formulation as prepared in Example 1 is more effective in reducing mean 24 hour systolic blood pressure (SBP) and diastolic blood pressure (DBP) as compared to valsartan immediate release formulation Diovan in hypertensive patients. Particularly, the blood pressure control observed over the 20-24 hour period after administration of valsartan ER formulation was significantly better than that obtained with Diovan®. The 20 to 24 hour period being the period where maximum adverse cardiovascular events occur, the protection provided during this period by administration of valsartan ER formulation is of significance. Treatment of hypertension with valsartan extended release formulation therefore offers complete 24 hour blood pressure control with adequate control during the 20-24 hour time period after administration avoiding gaps in treatment observed with conventional immediate release Diovan®.

1. A method of treating hypertension comprising administering once a day to a patient in need thereof the compound valsartan in a gastroretentive an extended release dosage form
wherein the gastroretentive extended release dosage form comprises:
a gastroretentive core comprising
(a) a therapeutically effective amount of valsartan of about 160 mg;
(b1) alpha-tocopherol polyethylene glycol succinate in an amount of about 80 mg;
(b2) polyoxyethylene polyoxypropylene block copolymer in an amount of about 80 mg;
(c1) polyethylene oxide in an amount of about 120 mg;
(c2) hydroxypropyl methylcellulose in an amount of about 230 mg;
(c3) hydroxyethylcellulose in an amount of about 60 mg;
(d) cross-linked polyvinyl pyrrolidone in an amount of about 150 mg;
(e) dextrates in an amount of about 60 mg; and
(f) a pharmaceutically acceptable carrier; and
at least one coating layer surrounding the gastroretentive core;
wherein the gastroretentive extended release dosage form reduces mean systolic and diastolic blood pressure in a patient during a time period of about 20 hours to about 24 hours after administration to a greater extent than an immediate release formulation of valsartan.

2. (canceled)

3. (canceled)

4. The method of claim 1 wherein the gastroretentive extended release dosage form comprises 160 mg of valsartan.

5. (canceled)

6. The method of claim 1 wherein the gastroretentive extended release dosage form comprises valsartan in a solubilized form.

7. A method of treating hypertension comprising administering once a day to a patient in need thereof the compound valsartan is a gastroretentive extended release dosage form
wherein the gastroretentive extended release dosage form comprises:
a gastroretentive core comprising
(a) a therapeutically effective amount of about 160 mg;
(b1) alpha-tocopherol polyethylene glycol succinate in an amount of about 80 mg;
(b2) polyoxyethylene polyoxypropylene block copolymer in an amount of about 80 mg;
(c1) polyethylene oxide in an amount of about 120 mg;
(c2) hydroxypropyl methylcellulose in an amount of about 230 mg;
(c3) hydroxyethylcellulose in an amount of about 60 mg;
(d) cross-linked polyvinyl pyrrolidone in an amount of about 150 mg;
(e) dextrates in an amount of about 60 mg; and
(f) a pharmaceutically acceptable carrier; and
at least one coating layer surrounding the gastroretentive core;
wherein the gastroretentive extended release dosage form exhibits more than 25% reduction in mean diastolic blood pressure when compared to an immediate release formulation of valsartan during the 20-24 hour time period after administration.

8. (canceled)

9. (canceled)

10. The method of claim 7 wherein the gastroretentive extended release dosage form comprises 160 mg of valsartan.

11. (canceled)

12. The method of claim 7 wherein the gastroretentive extended release dosage form comprises valsartan in a solubilized form.

13. A method of treating hypertension comprising administering once a day to a patient in need thereof the compound valsartan in a gastroretentive extended release dosage form
wherein the gastroretentive extended release dosage form comprises:
a gastroretentive core comprising
(a) a therapeutically effective amount of valsartan of about 160 mg;
(b1) alpha-tocopherol polyethylene glycol succinate in an amount of about 80 mg;
(b2) polyoxyethylene polyoxypropylene block copolymer in an amount of about 80 mg;
(c1) polyethylene oxide in an amount of about 120 mg;
(c2) hydroxypropyl methylcellulose in an amount of about 230 mg;
(c3) hydroxyethylcellulose in an amount of about 60 mg;
(d) cross-linked polyvinyl pyrrolidone in an amount of about 150 mg;
(e) dextrates in an amount of about 60 mg; and
(f) a pharmaceutically acceptable carrier; and
at least one coating layer surrounding the gastroretentive core; and
wherein the gastroretentive extended release valsartan dosage form exhibits more than 2 mmHg reduction in mean systolic and diastolic blood pressure when compared to placebo formulation during the 24 hour dosing interval.

14. The method of claim 13 wherein the gastroretentive extended release valsartan dosage form exhibits more than 3 mmHg reduction in mean systolic and diastolic blood pressure when compared to placebo formulation during the 20-24 hour period after administration.

15. The method of claim 13 wherein the gastroretentive extended release valsartan dosage form exhibits more than 4 mmHg reduction in mean systolic blood pressure when compared to placebo formulation during 20-24 hour period after administration.

16. A method of treating hypertension comprising administering once a day to a patient in need thereof the compound valsartan in a gastroretentive extended release dosage form
wherein the gastroretentive extended release dosage form comprises:
a gastroretentive core comprising
(a) a therapeutically effective amount of valsartan of about 160 mg;
(b 1) alpha-tocopherol polyethylene glycol succinate in an amount of about 80 mg;
(b2) polyoxyethylene polyoxypropylene block copolymer in an amount of about 80 mg;
(c1) polyethylene oxide in an amount of about 120 mg;
(c2) hydroxypropyl methylcellulose in an amount of about 230 mg;
(c3) hydroxyethylcellulose in an amount of about 60 mg;
(d) cross-linked polyvinyl pyrrolidone in an amount of about 150 mg;
(e) dextrates in an amount of about 60 mg; and
(f) a pharmaceutically acceptable carrier; and
at least one coating layer surrounding the gastroretentive core;
wherein the gastroretentive extended release valsartan dosage form exhibits more than 3 mmHg reduction in mean systolic and diastolic blood pressure when compared to placebo formulation during the 20-24 hour dosing interval; and wherein the gastroretentive extended release dosage form provides plasma concentration of valsartan greater than that provided by an immediate release formulation of valsartan over a time period of 20 to 24 hours after administration in a single dose human pharmacokinetic study.

17. The method of claim 16 wherein the gastroretentive extended release dosage form comprises about 40 to about 640 mg of valsartan.

18. The method of claim 16 wherein the gastroretentive extended release dosage form comprises 80 mg of valsartan.

19. The method of claim 16 wherein the gastroretentive extended release dosage form comprises 160 mg of valsartan.

20. (canceled)

21. The method of claim 16 wherein the gastroretentive extended release dosage form comprises valsartan in a solubilized form.
